(19) United States
(12) Patent Application Publication
(10) Pub. No.: US 2023/0032131 A1
(43) Pub. Date: Feb. 2, 2023

(54) DYNAMIC USER RESPONSE DATA COLLECTION METHOD

(71) Applicant: Limbic Limited, London (GB)

(72) Inventors: Ross Harper, London (GB); Sebastiaan De Vries, London (GB); Joshua Southern, London (GB)

(21) Appl. No.: 17/791,520

(22) PCT Filed: Jan. 8, 2021
(86) PCT No.: PCT/GB2021/050055

§ 371 Date: Jul. 7, 2022

(30) Foreign Application Priority Data
Jan. 8, 2020 (GB) 2000242.4

(51) Int. Cl. A61B 5/16; A61B 5/00; G16H 40/67

(52) U.S. Cl. CPC A61B 5/165; A61B 5/7264; A61B 5/7221; G16H 40/67

(57) ABSTRACT

The present invention relates to a method of continuously monitoring the biometrics of a user in order to determine and gather more accurate data on their mental state. More particularly, the present invention relates to a method of dynamically prompting the user in response to the monitoring of the user and the determination of the user's mental state. Aspects and/or embodiments seek to provide a method of substantially continuous monitoring of a user's mental state, and prompting of users based on their monitored mental state.

### FIELD OF THE INVENTION

The present invention relates to a method of continuously monitoring the biometrics of a user in order to determine and gather more accurate data on their mental state. More particularly, the present invention relates to a method of dynamically prompting the user in response to the monitoring of the user and the determination of the user's mental state.

### BACKGROUND

Mental illness is widely considered to be a growing problem economically and socially.

Depression, as one example of mental illness, can to varying degrees affect patient behaviour, thoughts, feelings, and sense of well-being and can be caused by one or more of a number of factors, such as biological genetics, the patient's environment, and psychological factors. Symptoms of depression can include “low” moods and an aversion to physical activity.

Economically, estimates of the annual costs of lost productivity due to depression and anxiety, and estimates of the financial costs of anti-depressants, amount to billions of dollars worldwide.

Although effective treatments are available for at least some patients, many individuals with depression either do not have access to such treatment or are averse to treatment due to lack of knowledge or information about their state and well-being, or are averse to treatment due to social stigmas surrounding such issues. This may result in fatal consequences for some untreated patients with serious conditions and, for those with less serious conditions, significant amounts of productive time lost due to mental illnesses and inefficient treatment processes.

Conventionally, for treatment of mental health issues, patients are directed to clinicians or mental health professionals for discussion of the various possible causes, and surrounding issues, in search for possible solutions. This process typically relies on patients self-reporting symptoms and information in relation to their own mental state, but such self-reporting is not always accurate due to it not being done, not being done comprehensively, or being done in an inaccurate fashion. Clinicians can therefore lack accurate data to discuss with patients and to determine the underlying problems or factors causing mental health issues, commonly resulting in a trial and error approach being adopted where patients try different methods of treatments in turn and the results are used to inform further treatment decisions or recommendations.

Studies have shown that patients generate fewer self-reports if not prompted or reminded, but if prompted at the wrong times can log irrelevant data or may become frustrated and not log their data at all. Importantly, self-reported data can be highly biased as it is not routinely captured or logged promptly and when logged after some time has passed subject to the quality of recollection of the patient, which is unlikely to be optimal.

Thus, it is necessary for new data driven methods suitable for monitoring mental states of patients in order to more efficiently report and diagnose any relating issues.

### SUMMARY

Aspects and/or embodiments seek to provide a method of substantially continuous monitoring of a user's mental state, and prompting of users based on their monitored mental state.

According to a first aspect, there is provided a method of prompting a user based on a determined mental state of a user, the method comprising the steps of: receiving user biometric data; determining at least one mental state of the user based on the received user biometric data; and on determining the at least one mental state of the user is a predetermined mental state, outputting one or more dynamic prompts for display to the user based on the at least one mental state of the user.

A system that monitors the mental and/or emotional state of a patient or a user using biometric data (such as data obtained from their wearable device) can be used to dynamically prompt the patient, for example, to input information that would normally only be collected via manual self-reporting or to display patient information to a user in certain situations or circumstances. This emotional/mental state of a patient, or user-input information/user response data, can be analysed or collected by a computer system and then made visible to a mental health professional or clinician, for example along with any analysis performed and/or any prompts shown to the user along with time stamp data.

Optionally, the step of receiving user biometric data comprises receiving user biometric data from any or any combination of: a wearable device of the user; images of the user; user speech data; or user text data.

User wearable devices, such as smart watches or fitness trackers, can be used to substantially continuously measure biometric data of a user, alongside other contributing variables to states of mental health, which can allow a wider system to proactively interact with patients, for example through a patient software interface or software application, at times most relevant to their mental health. The software interface or software application can be provided on the wearable device or via another user device such as their smartphone, tablet computer, laptop computer or desktop computer either via a dedicate software application or a web interface accessible using a web browser.

Emotion recognition, mental state recognition, or identifying a user's emotional state and/or mental state, can be also or alternatively be performed by analysing the user's facial expressions using image data for example. Facial recognition for the purposes of emotion detection and/or mental state detection can be implemented for example by comparing facial features of the user from an image or video obtained from the imaging device. Audio data or voice recognition can also be used to identify the user's emotional state and/or mental state for example by analysing the user's verbal expressions or verbal content.

Optionally, the method further comprises the step of receiving user response data entered by the user in response to the one or more dynamic prompts.

By monitoring the emotional state and/or mental of the patient or user, the system can detect states of and/or changes in user emotional state and/or mental state and some or all of these changes can be used to dynamically prompt the patient to journal, record their mood, log or self-report. Prompting a user in this way can result in the user providing relevant self-reporting data at, or shortly after, a change in mental and/or emotional state or the detection of a mental and/or emotional state.

Optionally, the at least one mental state of the user comprises any one or more of: at least one emotional state a range of emotional state; a range of emotional states; a range of mental state; a range of mental states; a probability score, optionally of a given mental state and/or emotional state; a confidence value, optionally of a given mental state and/or emotional state; one or more confidence values, optionally of a given mental state and/or emotional state; a probability distribution of confidence values, optionally of a given mental state and/or emotional state; one or more clinical scores of emotional state; one or more clinical scores of mental state; one or more clinical scores of mental illness; a PHQ-9 score; a GAD-7 score; one or more cognitive markers of depression and/or anxiety; any other clinical measure(s) of mental illness. Optionally, the supplementary and/or continuous data is received in substantially real-time. Optionally, the step of determining the at least one mental state of the user comprises determining one or more weightings for the user biometric data.

The mental and/or emotional state of the user can be determined with a probability/confidence for the detected mental and/or emotional state, which may be utilised by the system when it determines whether to dynamically prompt the user (e.g. if there is only a low confidence score for the determined emotional state, the system can be configured not to prompt the user below a certain confidence and/or probability threshold to avoid sending too many prompts to a user from the system and/or to avoid gathering low relevance data). The mental state and/or emotional state of the user can be determined substantially in real-time which can allow for the dynamic prompts to be sent to the user, for example while it is still relevant to collect self-reporting data from that user. The mental state and/or emotional state of the user can be determined using, for example, a learned algorithm for which weightings for factors/derived data/raw data can be determined for the user biometric data gathered for the user.

Although biometric data includes measurements of various physical properties in relation to the user, data gathered for the user may also include supplementary data which can be used to correlate with the biometric data to more accurately determine the user's mental state and/or emotional state. For example, the biometric data gathered might include heart rate data from a wearable device while the supplementary data might include the current typing speed of the user on another device such as the user's laptop or smartphone.

If confidence scores/values are generated, then these can be included along with the measure of mental state of the user, and can be useful to determine whether to flag certain data to a clinician, and/or take further actions and/or display information to a user and/or request user provided data, optionally based on a predetermined threshold of confidence score/value.

If clinical scores, such as PHQ-9 or GAD-7 scores, are useful to collect, generate or determine, aspect and/or embodiments can generate these to allow easy use of the output of the system, for example by a clinician/professional when working with the user/patient.

Optionally, the at least one mental state of the user is determined using a computer based model: optionally the computer based model comprising one or more machine learning algorithms, wherein the one or more machine learning algorithms process any or any combination of: user biometric data; supplementary data; continuous data; speech; and/or text.

Through the use of learned models and/or machine learning approaches/algorithms, complex mental state and/or emotional detection models and/or dynamic prompting models can be generated and refined. Further, through the use of learned models and/or machine learning approaches/algorithms, responses to the dynamic prompts can be used to further train models in order to create a system tailored to, or more accurate for, each user or groups of users/all users.

According to a second aspect, there is provided a method for prompting a user based on a determined at least one mental state of the user, wherein the at least one mental state of the user is determined using user biometric data, the method comprising the steps of: receiving one or more dynamic prompts from a server system, wherein the one or more dynamic prompts is based on at least one predetermined mental state of the user; notifying a user with one or more dynamic prompts; and transmitting the user input to the server system.

Dynamically prompting a user on a user device in response to detected mental and/or emotional states of the user can allow the user to be prompted for an in return provide self-reporting information (for example in relation to their mental health) and make it easier for patients to provide high-quality data to their clinician (thus allowing for a framework for treatment-response evaluation). This can potentially reduce wasted time in therapy and potentially avoid trial and error treatment approaches, delay(s), and/or the risk of premature discharge. It can also allow users to be notified with pertinent information in response to one or more certain detected emotions.

Optionally, the user biometric data comprises data from any or any combination of: a wearable device of the user; images of the user; user speech data; or user text data.

Optionally, the step of notifying the user comprises notifying the user to provide user response data via a user device; then the further steps of: receiving user response data via the user device in response to notifying the user; and transmitting the user response data to the server system, optionally wherein the user response data comprises one or more user inputs.

The dynamic prompt can be displayed to a user on a user device such as a smartphone or wearable device, and require a response from the user specific to the dynamic prompt such as to complete a self-reporting entry or to complete a questionnaire.

Optionally, the user biometric data comprises supplementary and/or continuous data. Optionally, the supplementary data is received from a secondary user device and wherein the supplementary data comprises any one or more of: mobile data; geo location data; mobile usage data; typing speed; or accelerometer data. Optionally, the continuous data comprises any one or more of: heart data; peripheral skin temperature data; Galvanic Skin Response (GSR) data; location data. Optionally, the user biometric data further comprises any one or more of: sleep data; activity data; historical emotional states; historic mental states.

User devices such as wearable devices (e.g. smart watches or fitness trackers) can be used to substantially continuously measure the biometric data of a user, as well as other variables that can contribute to the measurement of states of mental health, which allows the system to proactively interact with patients, for example through a patient software interface or software application, at times determined to be most relevant to their mental health. The software interface or software application can be provided on the wearable device or via another user device such as their smartphone, tablet computer, laptop computer or desktop computer either via a dedicate software application or a web interface accessible using a web browser. Similarly, supplementary data from these other user devices can be collected to form a more complete dataset, including both biometric and supplementary data for each user.

Optionally, the one or more dynamic prompts comprise any or any combination of: mood based prompts; time based prompts; location based prompts; people based prompts; response triggering prompts, optionally wherein the response triggering prompts comprise requesting the user to provide user response data.

The dynamic prompts shown to the user that are generated/triggered by the system can be based on a variety of factors and can request different sets of information from the user depending on the factors involved and the determined emotional state of the user. The emotional state of the user can be determined to be one or more of a variety or combination of states. The prompts can also require the user to provide user response data.

Optionally, the at least one mental state of the user is substantially discrete: optionally wherein the at least one mental state of the user comprises any one or more of: happy; sad; pleasure; fear; anger; hostility; calmness; excitement; and/or any other psychological and/or mental and/or emotional state relevant to the mental health of the user.

Optionally, in addition to the at least one mental state of the user, a mental health condition of the user can be determined; further optionally wherein the mental health condition comprises any or any combination of: depressed; anxious; bipolar; manic; and/or psychotic.

Optionally, one or more user interfaces are used, wherein the one or more user interfaces displays one or more of: the at least one pre-determined mental state of the user; at least one pre-determine emotional state of the user; user biometric data comprising at least heart data; the one or more user inputs; the one or more dynamic prompts; the mental state of the user and/or the emotional state of the user.

Optionally, the user input is provided through the one or more user interfaces. Optionally, the one or more user interfaces is in communication with a remote system. Optionally, further comprising a step of determining one or more recommendations based on the determined emotional state of the user.

A system that monitors the mental and/or emotional state of a patient or a user using their wearable device can be used to prompt the patient to input information that would normally only be collected via manual self-reporting. This patient or user-inputted information to be analysed is collected by a computer system and then made visible to a mental health professional or clinician along with any analysis performed. The platform can have a variety of user interfaces, depending on the device used by the user to view data from the platform or input data to the platform, and/or depending on the detected emotions/mental states of the user.

Optionally, there is a further step performed comprising the step of training a computer-based model for determining the mental state of the user based on any one or more of: the user biometric data; the one or more user inputs; the emotional state of the user; the at least one pre-determined mental state of the user and/or the at least one pre-determined emotional state of the user.

Through the use of learned models and/or machine learning approaches/algorithms, complex emotional/mental state detection models and/or dynamic prompting models can be generated and refined. Further, through the use of learned models and/or machine learning approaches/algorithms, responses to the dynamic prompts can be used to further train models in order to create a system tailored to, or more accurate for, each user or groups of users/all users.

Optionally, the step of determining at least one mental state of the user based on the received user biometric data further comprises determining at least one associated confidence value. Optionally, the step of outputting one or more dynamic prompts for display to the user based on the at least one mental state of the user comprises outputting one or more dynamic prompts for display to the user based on the at least one mental state of the user and one of the at least one associated confidence values.

Determining one or more confidence values associated with the determine one or more mental states of a user can allow for more discretion as to whether to take further actions based on the determined one or more mental states and/or whether to flag and/or report the determined one or more mental states (for example to a clinician or professional assisting the user/patient). The combination of the determined mental state(s) and the associated confidence value(s) can be used to determine whether to prompt a user, as for some mental states a lower confidence score might be suitable but some other mental states only a high confidence may be suitable to display prompts, or even certain prompts, to a user.

Optionally, any of the steps of: (a) determining at least one mental state of the user based on the received user biometric data; and/or (b) on determining the at least one mental state of the user is a predetermined mental state, outputting one or more dynamic prompts for display to the user based on the at least one mental state of the user; comprise using one or more probabilistic models. Optionally, the method further comprises using one or more probabilistic models. Optionally, the one or more probabilistic models comprise any or any combination of: a Bayesian deep neural network incorporating Monte Carlo dropout or variational Bayes methods to approximate a probability distribution over one or more outputs; a hidden Markov model; a Gaussian process; a naïve Bayes classifier; a probabilistic graphical model;

a linear discriminant analysis model; a latent variable model; a Gaussian mixture model; a factor analysis model; an independent component analysis model; and/or any other probabilistic machine learning method/technique that generates a probability distribution over its output.

Using probabilistic models can allow the determination of a confidence value for the determined one or more mental states, and various probabilistic models can be used.

For example, a dynamic prompt such as displaying a cognitive behavioral therapy (CBT) exercise to a user can be based on a combination of the inferred mental state and the model's confidence in that inferred mental state.

Optionally, the step of outputting one or more dynamic prompts for display to the user based on the at least one mental state of the user is dependent on a combination of the at least one mental state of the user and the at least one associated confidence values of the at least one mental state.

Optionally, the at least one associated confidence values of the at least one mental states exceeds one or more predetermined thresholds.

By using the combined predicted mental state(s) and associated confidence values, a more reliable prompting of the user can be achieved. For example, by using a threshold against which the confidence value can be checked, predicted mental state(s) with a confidence value below the threshold can avoid triggering a dynamic prompt to the user to ensure that only higher confidence predictions of mental states are used by the system to prompt the user.

For example, the model may predict symptoms of depression and/or low mood in a user, however the model confidence in that prediction is below a threshold for the system to take any further action, and so no dynamic prompting of the user will occur.

In another example, the model may predict symptoms of depression and/or low mood in a user, and the model confidence is above the threshold for the system to take a further action, thus the system consequently might aim to collect clinically meaningful data from the user (for example, associated thoughts, feelings, and behaviours) in order to gather the most relevant high-quality information for use in a clinical setting. For example, the system might dynamically prompt the user to complete a relevant questionnaire, or complete a journal entry, or use a chat interface to ask a series of questions (for example using a decision tree approach to generating the sequence of questions, or using one of a predetermined standard set(s) of questions).

Optionally, even where a low confidence is output and this is below the threshold, the system may continue to collect clinically meaningful data from the user but might apply a lower weighting or lower significance to the collected user data as this can tune the relevance of the clinical data collected based on the model's confidence in the prediction of the user mental state(s) at the time of collection.

Optionally, the user response data comprises any or any combination of: information for use in a clinical setting; clinically meaningful data from the user, optionally comprising any or any combination of associated thoughts, feelings and/or behaviours; data gathered at clinically salient moments; data gathered within a predetermined time of the detected at least one mental states; associated relevant data, optionally comprising one or more associated confidence values.

The dynamic prompts of the system to the user can be tailored to supply and/or retrieve clinically meaningful information based on the real time inference of the user/patient's mental state(s), optionally based on the confidence of the model for these mental state(s) inference(s). From a retrieval perspective, for clinicians and/or professionals assisting the user/patient, it can be critical to gather user input data on their thoughts, feelings, and behaviours at times when they are exibiting certain, or the strongest, mental state(s) and/or emotion(s) or cognitive markers of mental illness (such as PHQ-9 and GAD-7 scores). By collecting this information during clinically salient moments, i.e. within a certain time of the inference of the relevant emotion(s)/mental state(s), the resulting user response data can be more informative for psychological therapy. From a supply perspective, i.e. from the perspective of what information and/or support is offered to the user/patient, it can be important to supply digital content which is relevant for the emotional state/mental state/current symptoms of mental illness that the user/patient is currently experiencing, such as a guided cognitive behavioral therapy exercise aimed at treating depression and/or a journal such as a gratitude journal and/or a breathing exercise or other exercise that can be dynamic based on the data from the user devices and/or the inferred mental state/emotions of the user.

Optionally, the associated relevant data is used to assign a weighting and/or importance to the user response data.

Where confidence values for the inferred/determined mental state(s) and/or emotion(s) are low, then a lower weighting or importance can be assigned to that inference and/or any user response data and vice versa where confidence values are high then a higher weighting or importance can be assigned to the same.

Optionally, the method further comprises a step of performing statistical analysis on the relationships between the user response data and the determined at least one mental states, optionally outputting one or more measures of the relationships between the user response data and the determined at least one mental states.

By performing a statistical analysis on the relationships between the user response data and the determined at least one mental states, the statistical analysis can be output to the user and/or a clinician/professional to feed into standard behavioral psychology techniques and/or can be used to implement automated assistance or content selection to display to the user or cause the user to interact with.

Optionally, outputting one or more dynamic prompts for display to the user comprises outputting one or more dynamic prompts supplying content to the user determined to be relevant to the determined at least one mental state of the user; and wherein the user response data is collected following the content being supplied to the user.

Sometimes it can assist the user/patient to be displayed relevant content such as video content, audio content, or text content that is relevant to the detected/predicted/inferred mental/emotional state(s), following which any interactive process such as collecting data and/or interactive exercises based on user responses/biometric/etc data can be performed. This can allow the patient to receive assistance with the determined mental state/emotional state following which clinically meaningful information can be gathered. Alternatively, the clinically meaningful information can be gathered first and then any content can be displayed to the user, optionally where the clinically meaningful information can be used as part of the determination of what content to be displayed to the user.

It should be appreciated that many other features, applications, embodiments, and variations of the disclosed technology will be apparent from the accompanying drawings and from the following detailed description. Additional and alternative implementations of the structures, systems, non-transitory computer readable media, and methods described herein can be employed without departing from the principles of the disclosed technology.

### BRIEF DESCRIPTION OF DRAWINGS

Embodiments will now be described, by way of example only and with reference to the accompanying drawings having like-reference numerals, in which:

FIG. 1 shows an illustration of traditional interactions between patients and mental health professionals;

FIG. 2 shows an illustration of an embodiment described herein showing an overview of the system, patient wearable device, patient user interface deployed on a patient device, and clinician user interface;

FIG. 3 shows an illustration of an example patient user interface;

FIG. 4 shows an illustration of an example clinician user interface;

FIG. 5 shows a flowchart depicting the underlying process within the cloud system;

FIG. 6 shows a flowchart depicting the process of self-reporting by responding to dynamic prompts output from the cloud; and

FIG. 7 shows a process according to an embodiment using a user device, some extracted input data which is pre-processed and then input to a Bayesian neural network, the outputs of which are then used to show a prompt to a user on the user device.

The figures depict various embodiments of the disclosed technology for purposes of illustration only, wherein the figures use like reference numerals to identify like elements. One skilled in the art will readily recognize from the following discussion that alternative embodiments of the structures and methods illustrated in the figures can be employed without departing from the principles of the disclosed technology described herein.

### DETAILED DESCRIPTION

Data is used to support clinicians in a variety of medical sectors. However, more and better data needs to be provided to support clinicians practicing in the mental health field. Currently, mental health professionals rely almost exclusively on patients to provide self-reporting information. However, typically patient self-reporting information is collected both infrequently and is potentially unreliable, but this information is required for accurate assessment and analysis of any mental health issues and to assess treatment options. In particular, where sufficient and/or reliable data is absent, clinicians are typically forced to adopt trial-and-error techniques to attempt to find mental health treatments that work for each patient. To guide their decision-making, especially where self-reporting information is absent or poor, clinicians and mental health professionals need to spend significant amounts of time in consultations with patients trying to acquire the information they need to perform an assessment of mental health. In contrast, the number of consultations, and amount of time required for each consultation, for patients who provide high quality self-reporting information can be significantly fewer/lower than for patients where little or no self-reporting data is available. Therefore, the problem of absent or low quality patient data needs to be addressed in order to make assessment of mental health issues more efficient and to enable clinicians to effectively treat mental health issues, preferably at the early stages of such mental health issues occurring.

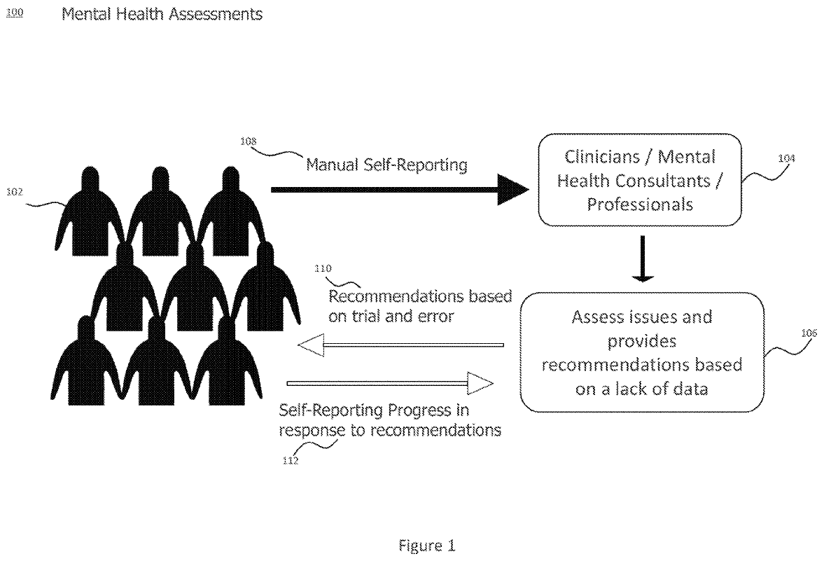

FIG. 1 illustrates the typical problems of traditional methods of communication 100 between patients and mental health professionals during typical mental health treatment.

As shown in FIG. 1, patients 102 are provided with paper forms or a diary, or digital forms or digital diary, to record self-reporting information 108 that the clinician considers to be relevant, or simply to record a standard variety of information (e.g. answer a standard set of questions). The manual self-reporting information 108 is provided to the clinician 104 by the patient 102, usually by giving it to the clinician 104 in person at an in-person appointment. The clinician 104 will then assess 106 the patient 102 using any self-reporting information 108 provided by the patient 102. However, typically, the patients 102 provide little or unreliable self-reporting information 108. Through these current approaches of using self-reporting 108, mental health professionals 104 are typically not supported with sufficient data nor is the quality of the data received from patients 102 sufficient to promptly and efficiently provide methods of treatment to patients 102. Recommendations 110 will then be provided by the clinician 104 to the best of their ability based on the data and information available. The patients 102 then provide (or not) self-reporting progress information 112 for the clinician 104 to re-assess 106 any recommendations.

Cognitive Behavioural therapy (CBT) is a form of psychotherapy, specifically a psycho-social intervention, that aims to improve mental health and which focuses on challenging and changing unhelpful cognitive distortions and behaviours in patients, improving their emotional regulation, and helping patients develop personal coping strategies. CBT is the most used psychotherapy in UK, and potentially worldwide. In order to perform CBT effectively, it is important that patients regularly and promptly perform certain actions including for example logging their mood, and logging who they are with when experiencing a certain emotional state.

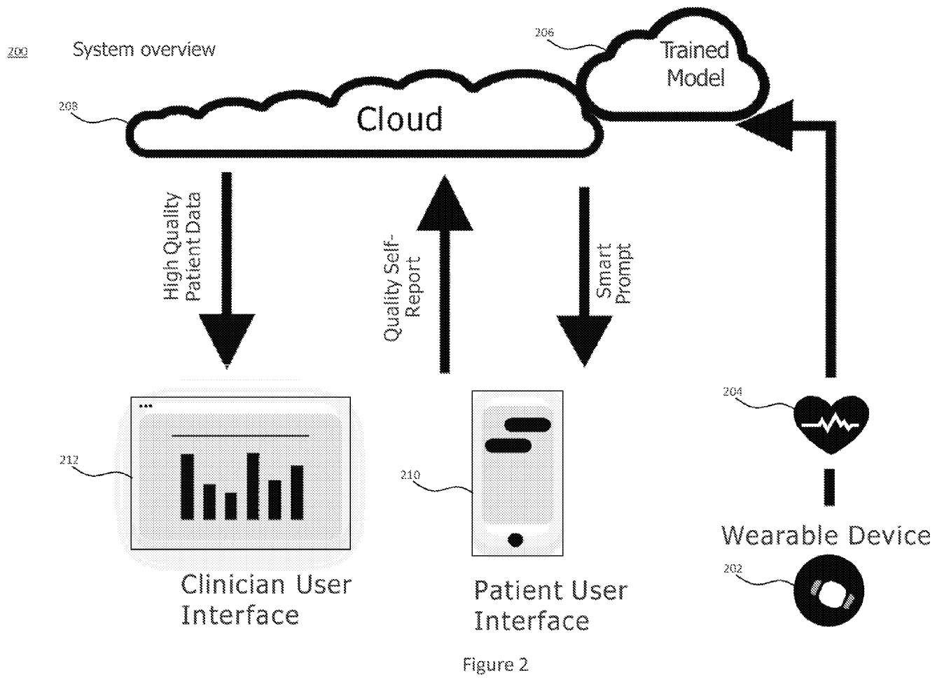

With reference to FIGS. 2 to 6, embodiments setting out an example method of dynamically prompting a user, for example to provide self-reporting information, by monitoring a user's mental state, will now be described.

Example embodiments described herein provide a patient/user reporting platform that uses learned algorithms to process information obtained from a user, either via prompting a user to provide information or by monitoring the biometrics/physiology of a user (for example, via their wearable device), to generate high quality information for analysis by a computer system and/or a mental health professional or clinician. In example embodiments herein, although the output is referred to as the detection or monitoring of emotional states, the output may also be a mental state or the combination of emotional state(s) and mental state(s). Although there may be a correlation between a user's emotional state and their mental state, an emotional state does not provide a clinical measure of mental illness. A mental state on the other hand may constitute a clinical measure, for example using a scoring-based system such as a “PHQ-9 score” (which is a 9 question survey that measures symptoms of depression) or a “GAD-7 score” (which is a 7 question survey that measures symptoms of anxiety). Thus, in some embodiments, a mental state can complement the emotional state prediction and/or be determined in absence of emotional state prediction.

Computer-based models can detect emotional states and/or mental states and changes in physiology that cannot be explained by elevated activity (e.g. exercise), and in the described embodiments these detected changes are used to prompt the patient (for example to provide information; to complete a journal entry; to record their mood; to log certain information; or to otherwise keep their self-reporting up to date with substantially real-time information). Such models can be learned, trained or developed for use with consumer wearable devices, for example, or with other user devices such as a mobile phone. The output detected emotional states and/or mental states from these computer-based models can be used to dynamically prompt the patient to provide information at detected relevant times (e.g. on or shortly following certain changes in emotional state or physiology). In some example embodiments, the dynamic prompts are for the user to input data that is relevant to the mental state of the patient as this is relevant to the clinical treatment of the patient. This information can then be collected from the user following the prompt, received by the system, then processed then stored ready to be displayed in some form to a clinician, making it easier for patients to provide high-quality data to their clinician. As the computer-based models learn on clinical data, the system can be extended to diagnosing mental illness, providing early-warning of acute psychiatric episodes, or providing a measure of mental health for a patient.

In this example embodiment, user emotional states and/or mental states are determined from a user wearable device obtaining biometric data from the user and a remote system processes this data to determine one or more emotional states and/or mental states of the user wearing the device.

Alternatively, in other embodiments, user emotional states and/or mental states can be determined using image or video data of the user, for example the face of the user, to detect as the facial expressions of the user changes.

Further, in other embodiments audio data, from audio or video recordings, can be used to determine the emotion of a user or their mental state.

In other embodiments, text data such as text in e-mails or messaging applications or software can be used to determine the emotional state and/or mental state of a user.

In still further embodiments, a combination of these data sources can be used to determine the emotion or mental state of a user.

FIG. 2 shows a system overview 200 of the method according to an example embodiment, which will now be described in more detail.

More specifically, this figure shows that a wearable device 202 is used to obtain biometric data, specifically heart data 204, of a wearer. The heart data 204 is transmitted to a remote system 208, at which there is a provided a trained computer-based model 206. The trained computer-based model 206 is a learned algorithm which is used to assess user emotional state and/or mental state (and is trained on biometric data, such as heart data from other users) using heart data 204 from the wearable device 202. In example embodiments, the wearable device 202 is used to continuously measure heart data of a wearer (in other embodiments, other biometric data can also be measured, where the wearable device 202 is provided with the sensing and processing capabilities to do so). A wearable device 202 can be, for example, a consumer device such as a fitness tracker or smart watch that is worn by a user and which is provided with sensors that can monitor, usually substantially continuously, the physiology of the wearer. These devices are provided with processing capabilities and can process the data collected by the sensors to at least a limited extent, for example to determine heart rate information or heartbeat dynamics. Such consumer devices are discrete and easy-to-use unlike expensive medical equipment used conventionally for mental health data extraction or data collection. In example embodiments, the use of a wearable device 202 or tracker allows clinicians to collect various user data other than patient self-reporting data, such as for example sleep data, activity data, heart data, heartbeat measurements, location data, and/or skin temperature data, which can be relevant to understanding both the mental health and general health of patients when considered in an appointment with a patient or when considering a patient.

The cloud system 208 can communicate with various devices providing user interfaces including those providing a patient user interface 210 and a clinician user interface 212. The cloud system 208 provides different information via the different user interfaces such as the patient interface 210 and the clinician interface 212 in order to provide relevant or requested data and can also receive responses and input via the user interfaces 210, 212. For example, the cloud system 208 in communication with the patient interface 210 can be tailored to prompt the user of the patient interface 210 to respond to dynamic, or “smart”, prompts for patient self-reporting based on the detected emotional state from the user's wearable device 202. Alternatively, or as well, in some embodiments the prompts can include questions to specifically determine the user's emotional state and/or mental state. The user's emotional and/or mental state can be assessed based on a series of questions which can be used to determine a clinical score to represent the user's mental state. Additionally, or alternatively, the prompts may also include information pertinent to the user based on the detected emotion of the user, for example information on breathing techniques if the user is determined to be stressed or angry. If only information is provided, then no response from the user may be collected and sent or just a confirmation that the user reads the information may be sent.

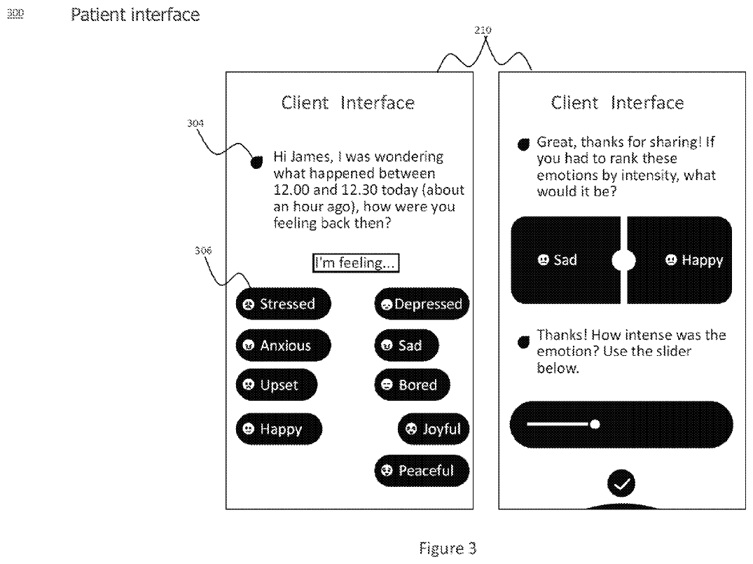

FIG. 3 shows an illustration 300 of an example patient user interface 210. The example client interface 210 illustrates a simple chat-based interface. Particularly, the patient user interface 210 can receive a dynamic prompt 304 from the cloud system 208 to prompt the patient when certain changes in emotional state and/or mental state, or certain emotional/mental states, are detected through the system monitoring the wearable device 202. Such states can be any psychological or emotional states relevant to their mental health/mental state. Such dynamic prompts can be immediate or slightly delayed following the relevant detection that triggers the system to send the dynamic prompts. Also, general prompts can be sent at regular intervals, for example once a week or month. The user is prompted through the user interface to answer straightforward or simple questions such as “how are you feeling?”, “what are you doing?”, or “who are you with?”. Options for answers can be presented as set responses 306, or free text can be entered (or voice or video responses recorded using the device on which the user interface 210 is presented). Such response prompting messages, whether text or speech or video based, can stimulate responses collecting information about people, location, mood(s), and/or time and follow on questions may request further information on the reasoning for the responses provided by the user (or optionally the user may comment on their responses).

In one implementation, the dynamic prompts 304 can require one of multiple set responses 306 that the user can choose from, to keep the user interface 210 simple and minimizing the user effort involved in providing self-reporting information via the user interface 210. The set responses 306 can alternatively be in the form of a multiple-choice question, a probability-based response (represented for example by a sliding scale that can be moved by a user from 1 to 100 or happy to sad), or a rating-based response (for example 1 to 5 stars out of 5) for example, although response options are not limited to these examples sometimes the prompt can only display information.

The patient user interface 210 can make self-reporting more accurate, as it can dynamically prompt the user at an appropriate time (e.g. immediately following detection of an emotion, or within a time period thereafter) and makes it easier for patients to provide self-reporting data in response, and so should result in more reliable mood logs and diaries of information for patients. The dynamic prompts 304 are determined by the computer-based models which identify emotional events using the wearable device. This allows the system to dynamically prompt the patient for information via the patient user interface 210 and removes the need for the patient to remember to record information either at set time intervals or in response to their mood or emotions.

In example embodiments, dynamic prompts shown to the user that are generated/triggered by the system can be based on a variety of factors and can request different sets of information from the user depending on the factors involved and the determined emotional/mental state of the user. For example, if through analysis of heartbeat dynamics or heart data the user is predicted to be in a state of anxiety (often characterised by high levels of arousal and low emotional valence), the system can prompt the user to provide information on contextual factors such as current location, environment, social factors, or other thoughts, feelings, and/or behaviours. By prompting the user during or near to the experienced state of anxiety, the self-reported information by the user will more likely be of high accuracy, quality and relevance for guiding the therapeutic processes. High quality self-reported data can be useful for the identification of external triggers of anxiety, or monitoring progress through treatment and/or recovery for example.

In some embodiments the dynamic prompts are not pre-timed or generic interactions, but instead refer specifically to the well-timed collection of clinical information necessary for psychological therapy. For example, the system can perform statistical analysis on the relationships between thoughts-feelings-behaviour, which is the bedrock of behavioural psychology techniques, after obtaining and sampling thoughts and feelings in substantially real-time.

In managing a user's emotional or mental state, it is critical to gather feedback from the users and particularly on their thoughts, feelings and behaviours at times when they are experiencing the strongest emotional state, or in response to cognitive markers of mental illness (such as inferred PHQ-9 and GAD-7 scores). By collecting this information during ‘clinically salient’ moments, the resulting user data is more informative for psychological therapy. It is important to supply digital therapeutic content via the user's device, which is relevant for the emotional state/mental state/current symptoms of mental illness that the user is currently experiencing. This too can be provided through dynamic prompts. For example, the trained model may infer (from any combination of sensor data, user input data or clinical data) that the user is experiencing symptoms of a mental state such as depression (as indicated by a high PHQ-9 score). As a result, the system will push a guided CBT exercise programme specifically aimed at treating depression (e.g. a gratitude journal or breathing exercise).

In some embodiments, an implementation can involve just the delivery of relevant information, rather than the collection of information from the user or patient. In this embodiment, analysis of heartbeat dynamics or heart data may for example lead to a prediction that the user is in a state of “low” mood such as a depressive state and provide the user with relevant information pertaining to that predicted mood or state. The prediction of psychological state or mental state can as in other embodiments be generated through heart data analysis as well as any one or more of the supplementary data received from the secondary user device including, however not limited to, mobile data, geo-location data, mobile usage data, typing speed, and/or accelerometer data, heat data, peripheral skin temperature data, and/or

Galvanic Skin Response (GSR) data.

The emotional state and/or mental state of the user can be a variety or combination of states and it may be difficult for users to swiftly determine how to overcome low moods or depression for example. Therefore, in example embodiments, based on a determination of mental state or prediction the system may provide the user with validated therapeutic information and/or tools to improve their mood such as encouraging the user to increase activity and exercise levels which can stimulate the body to produce natural anti-depressants, or reminding the user of techniques learned previously in therapy.

User biometric data received from the wearable device 202 can be complemented with other user data received from the wearable device or a secondary device such as a mobile device. This other user data can include, but is not limited to, supplementary data such as geolocation data, mobile usage data, typing speed, and/or accelerometery, and continuous data such as heart data, heat data, Galvanic Skin Response (GSR) data, and/or location data. Data obtained for the patient can be transmitted to the remote system 208 for analysis by the computer-based model(s) and/or for training of computer-based model(s). In example embodiments, physiological input signals may be captured by either or both of the wearable device and the secondary device and include a combination of galvanic skin response, heart data, pupillary response, skin temperature, respiration rate, and electroencephalogram data.

In some embodiments, the biometric data can be (or can be complemented with) image/video data obtained from an image sensor or imaging device. Image and video data can be used for facial recognition for example. Emotion recognition or identifying the user's emotional state can be typically determined by analysing the user's facial expressions. Facial recognition for the purposes of emotion detection can be implemented for example by a trained model comparing facial features of the user from an image or video obtained from the imaging device to training images or trained images stored in a database.

In some embodiments, the biometric data can be (or can be complemented with) audio data. Audio data can be used to identify the user's emotional state by analysing the user's verbal expressions for example. In human communication and interactions, non-verbal sounds within an utterance can also play important roles in determining and recognising emotional states. For example, non-verbal sounds such as laughter and cries naturally exist within conversations and can play an important role in accurately determining the user's state of emotion when complementing the user biometric data.

In some embodiments, the biometric data can be (or can be complemented with) text data. Text data can provide further context to determining the user's emotional state or can be used to determine the emotional state of the user. Words, phrases, and punctuation for example, as well as implied and explicit meaning/context, may be factored into the analysis and assessment of a user's emotional state.

The cloud system 208 can receive and process the patient or user data obtained via the patient user interface 210 or from the user's wearable device 202 into reporting information for on-demand display to a clinician, to enable the clinician to efficiently access the patient data that has been collected.

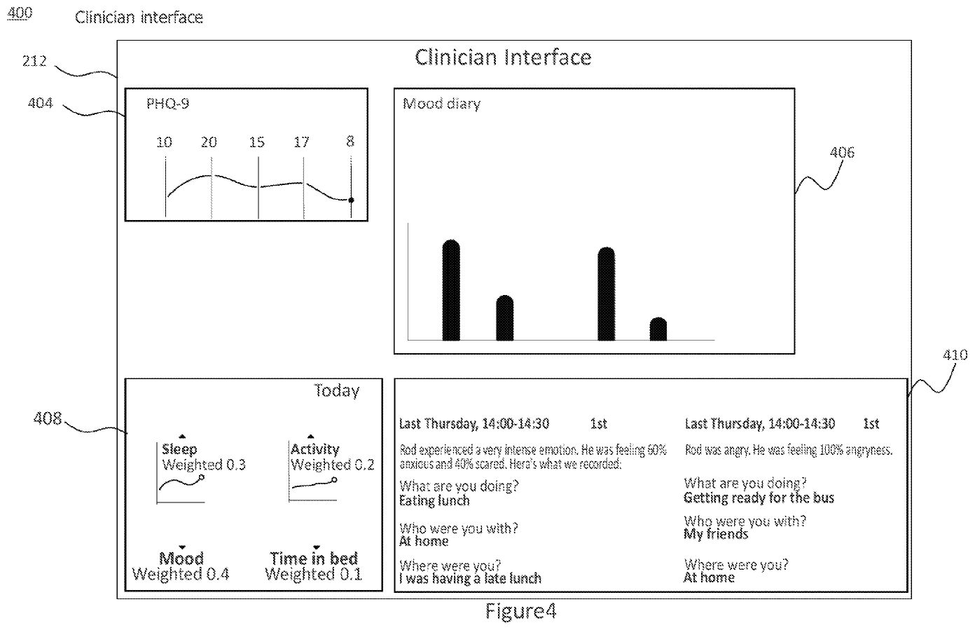

FIG. 4 shows an illustration 400 of an example clinician user interface 212 that allows the clinician to view this reporting information. The clinician interface can be provided as part of a mobile or web-based application and provides a clinician friendly display/interface of the data collected by the patient's wearable device and/or the patient user interface.

Patient self-reported data as well as measured data can be displayed in a simple format for the clinician to view before, during, and after consultations to assist the clinician. The clinician interface 212 is tailored for use by mental health professionals to provide emotional reports showing the results of long-term tracking of the relevant data for monitoring progression of the patient over time. The computer-based model used to detect patient emotion can also be trained to carry out various analysis of patient information for user friendly assessment of various factors contributing to patient mental health. Examples of displayed information may include weekly patient outcome measures 404, a mood diary 406, sleep quality and activity levels 408, and a collection of dynamic prompts and the responses input by the patient

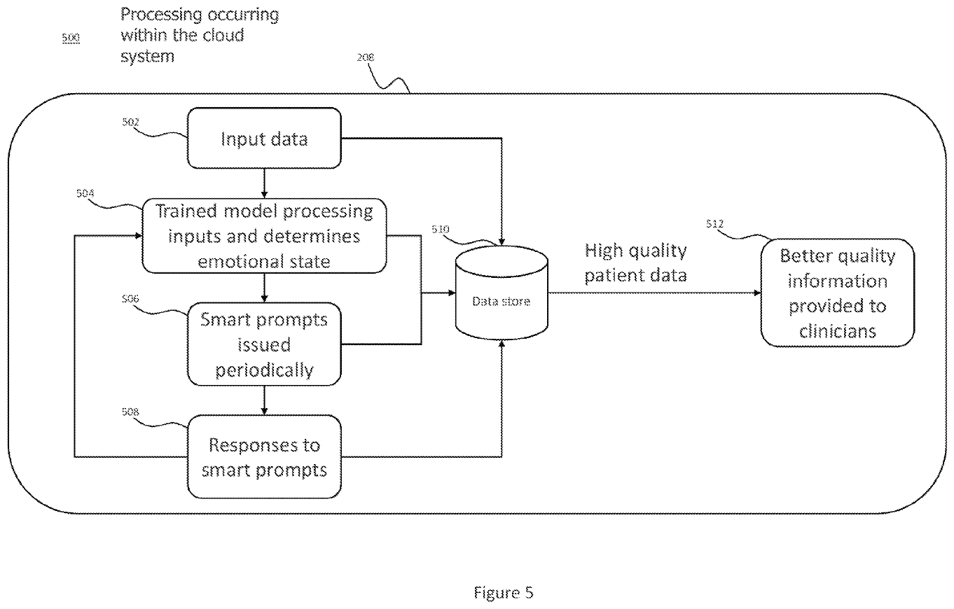

FIG. 5 shows a flowchart 500 depicting the underlying processing within the cloud system 208.

The cloud system 208 processes input data 502, which in this embodiment is biometric data received from the user's wearable device. In other embodiments, other data can be included in the input data 502, for example additional user data from a secondary device.

The input data 502 is provided to a trained model 504 that is suitable for processing user data to determine one or more emotional states. The output determined emotional state or states may also have an associated weighting or probability score, or a confidence score, in some embodiments. The cloud system 208 carries out background substantially continuous monitoring of user data from the wearable device and using the substantially continuous stream of data from the user's wearable device is substantially constantly assessing the user's mental condition or state using the trained computer-based models 504, such as a Bayesian Neural Network model, to predict emotional valence from heart data (as well as using other supplementary and continuous data obtained in accordance with sensor-availability in current consumer wearable devices and secondary devices, in some embodiments). Such computer-based models can use the latest advancements in time series modelling, such as recurrent and convolutional architectures, combined with probabilistic modelling to capture confidence in the output of the model which is specially trained for applications in healthcare.

In example embodiments, the trained models use probabilistic models which an allow the determination of a confidence value for one or more emotional and/or mental states. Various probabilistic models can be used to determine a confidence value of the determined output emotional and/or mental state. For example, the probabilistic models can be made up of any combination of a Bayesian deep neural network, using a Monte Carlo dropout technique (and using the Monte Carlo dropout technique to approximate a probability distribution over one or more outputs), a hidden Markov model, a Gaussian process, a naive Bayes classifier, a probabilistic graphical model, a linear discriminant analysis, a latent variable model, a

Gaussian mixture model, a factor analysis, an independent component analysis, and/or any other probabilistic machine learning method/technique that generates a probability distribution over its output.

Additionally, the computer-based model can be trained to output a dynamic prompt 506, otherwise known as a “smart” prompt, to the patient in order to capture substantially real-time responses 508 which may be relevant to the assessment of the user's mental state. In turn, the responses 508 to the dynamic prompts 506 can be used to further train the computer-based models 504 in order to create a more personal system tailored to the user. Moreover, all user data can be used to further train the computer-based models through speech and/or text processing. Thus, the computer-based model can in addition use speech and text data captured from the user to determine the user's mental state or emotions. A wealth of research within the field has focussed on analysis of natural languages for example and words can be extracted and their meaning inferred using machine learning tools, such as recurrent neural networks. In speech processing, intonation and speed also carries emotional information. The extracted words, inferred meaning(s), and other information derived by, for example, speech processing, can be used as inputs and training data for the models used.

Through the use of probabilistic models, the dynamic prompts can be dependent on a combination of the inferred emotional and/or mental state, and the confidence score for the inferred/determined state. For example, the model may predict symptoms of depression/low mood in a user, however, the confidence score for the output determined by the system is below a threshold for the system to act, and therefore does not send any prompts to the user. However, in some instances, even though the confidence in this emotional and/or mental state prediction is below a threshold, the system may continue to collect clinically meaningful data from the user (e.g. associated thoughts, feelings, and behaviours), which can be requested through the dynamic prompts.

In another example, when the system predicts symptoms of depression/low mood in a user, and the confidence score for the determined emotional and/or mental state is above the threshold for the system to act, the system consequently collects, through the dynamic prompts, clinically meaningful data from the user (e.g. associated thoughts, feelings, and behaviours) in order to gather the most relevant, high quality, information for use in a clinical setting.

The user data that is received in response to the dynamic prompts can be weighted based on the confidence score given to the determined emotional and/or mental state. For example, when user data is received when estimates with a confidence score below a certain threshold, it might down-weight the significance of the user data. The weighting of the user data can be used as a form of tuning the relevance of clinical data collected, based on the confidence score in the user's determined emotional and/or mental state at the time of collection.

The user input data 502, the continuously monitored emotional states from the trained models 504, the dynamic prompts 506, and the responses corresponding to the prompts 508 can all be stored in a data store 510 located in the cloud system 208 to be provided to a clinical or mental health professional on demand 512 or as set pieces of information (e.g. as a regular report). In this way, clinicians can obtain high-quality and more reliable patient data prior to consultations, for example.

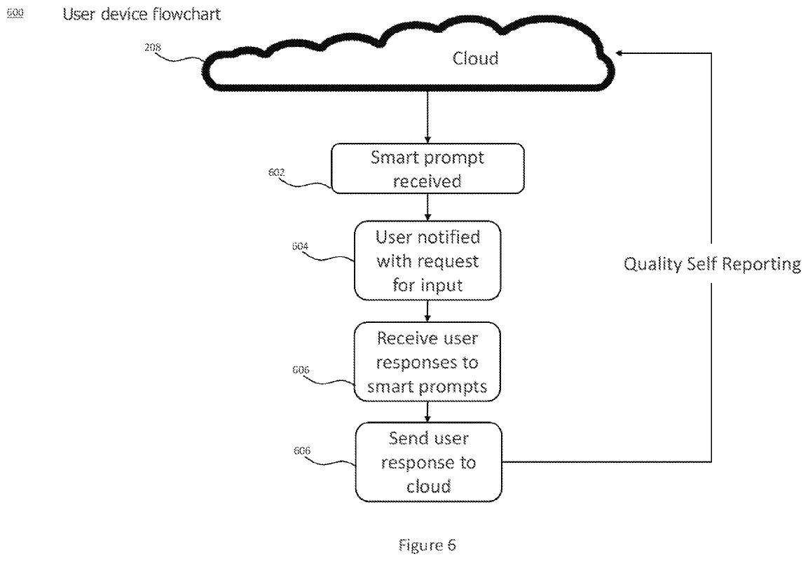

FIG. 6 shows a flowchart 600 depicting the process of self-reporting by a user responding to dynamic prompts output from the cloud system 208. In the example flowchart of FIG. 6, once a dynamic or smart prompt is received 602, the user is notified through the user interface (provided for example as an app on a smart phone or tablet, or a web page in a browser) with a request for response or input 604. The user device receives any user responses 606 to the smart prompts 602 and these are communicated to the cloud system 208 for further processing and/or analysis.

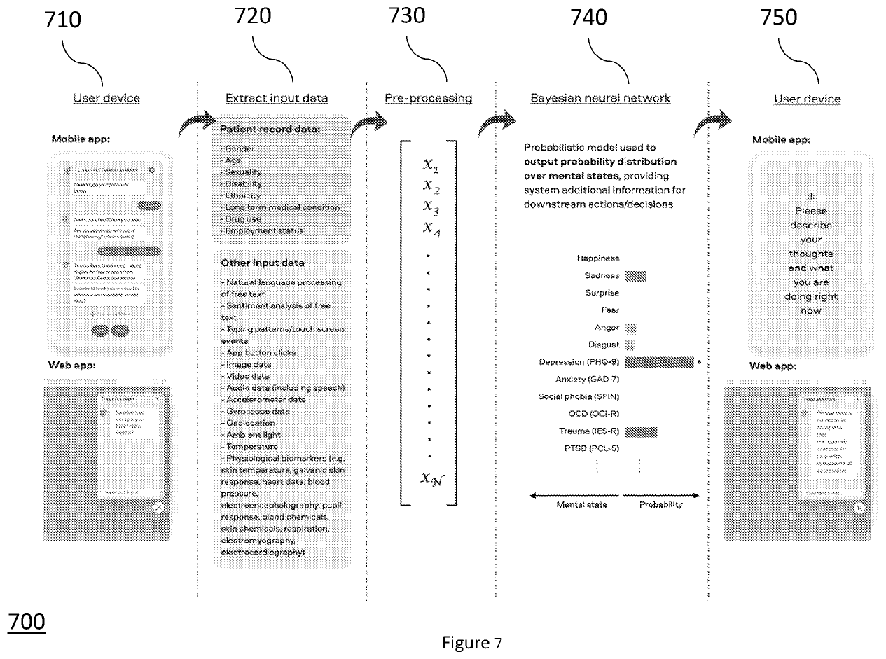

Referring now to FIG. 7, there is shown a process 700 according to an embodiment, where input data is pre-processed and a model used to infer mental state information and a prompt is shown to a user on the user device, and this will now be described in more detail below.

The user device can be many types of computer system, in this embodiment either a computer showing a web app or a mobile device showing a mobile app. In the web or mobile app, a chat interface is displayed 710, with the user entering data into the chat interface as needed.

Input data is extracted 720 from the user, either from patient record data (for example including in this embodiment gender, age, sexuality, disability, ethnicity, long term medical condition(s), drug use and employment status) and/or from other input data (for example including natural language processing of free text, sentiment analysis of free text, typing patterns, touch screen events, app button clicks, image data, video data, audio data (including speech), accelerometer data, gyroscope data, geolocation data, ambient light data, temperature data, and physiological biomarkers).

Natural language processing and sentiment analysis of free text, along with typing patterns can be applied to/used to assess text entered by the user into the chat interface of the mobile/web app but can also be used on text entered more generally on a user device. Touch screen events and app button clicks can be used where there is a suitable user device to gather this input data from. Depending on the sensors available, image data, video data, audio data (including speech), accelerometer data, gyroscope data, geolocation data, ambient light data, temperature data, and physiological biomarkers can be gathered as input data.

Physiological biomarkers can include any of skin temperature, galvanic skin response, heart data, blood chemicals, skin chemicals, respiration, electromyography and electrocardiography). Various combinations of this input data are possible in other embodiments, and may be dependent on the available devices and/or sensors pertaining to the user.

The pre-processing 730 can be performed on the input data to convert it and/or gather it into a suitable format for the trained models/probabilistic model(s) 740.

Next, the pre-processed data is received by a probabilistic model 740. Although this Figure illustrates a Bayesian neural network, as mentioned above, any type of probabilistic model can be used to output a probability distribution over emotional and/or mentals states. As depicted in this figure, the estimated state can be a range of emotional and/or mental states such as, happiness, sadness, surprise, fear, anger, disgust, depression (using PHQ-9), anxiety (using GAD-7), social phobia (using SPIN), OCD (using OCI-R), trauma (IES-R), PTSD (PCL-5), etc. This step may also include one or more scores for each state.

Finally, the user device via the mobile/web app interface 750 displays the output following the output based on the output probability distribution over mental states output by the neural network 740. Based on the scores or distribution of 740 the user can be presented with one or more dynamic prompts. The user may be prompted to provide clinically meaningful data from the user (e.g., associated thoughts, feelings, and behaviours). The emotional state of the user may refer to any psychological or emotional state relevant to the mental health of the user or symptomatic of a mental health condition, including any one or more of: happy; sad; pleasure; fear; anger; hostility; calmness and/or excitement.

A mental health condition may include any one or more of: depressed; anxious; bipolar; manic; and/or psychotic.

Biometric data may include any measurement of physical properties in relation to a user, and any calculated values derived therefrom. Such measurement of physical properties may include one or more of: heart rate; temperature; facial expression; skin moisture; breathing rate; and/or voice analysis.

In example embodiments, prediction of emotional states from multiple physiological signals can be implemented using machine learning models such as Naïve Bayes, Linear Discriminant Analysis, Support Vector Machine, Convolutional Neural Networks and Recurrent Neural Networks. Machine learning is the field of study where a computer or computers learn to perform classes of tasks using the feedback generated from the experience or data gathered that the machine learning process acquires during computer performance of those tasks. Typically, machine learning can be broadly classed as supervised and unsupervised approaches, although there are particular approaches such as reinforcement learning and semi-supervised learning which have special rules, techniques and/or approaches. Supervised machine learning is concerned with a computer learning one or more rules or functions to map between example inputs and desired outputs as predetermined by an operator or programmer, usually where a data set containing the inputs is labelled.

Unsupervised learning is concerned with determining a structure for input data, for example when performing pattern recognition, and typically uses unlabelled data sets. However, various hybrids of categories are possible, such as “semi-supervised” machine learning where a training data set has only been partially labelled. The use of unsupervised or semi-supervised machine learning approaches are sometimes used when labelled data is not readily available, or where the system generates new labelled data from unknown data given some initial seed labels.

Unsupervised machine learning is typically applied to solve problems where an unknown data structure might be present in the data. As the data is unlabelled, the machine learning process is required to operate to identify implicit relationships between the data for example by deriving a clustering metric based on internally derived information. For example, an unsupervised learning technique can be used to reduce the dimensionality of a data set and attempt to identify and model relationships between clusters in the data set, and can for example generate measures of cluster membership or identify hubs or nodes in or between clusters, for example using a technique referred to as weighted correlation network analysis, which can be applied to high-dimensional data sets, or using k-means clustering to cluster data by a measure of the Euclidean distance between each datum.

Semi-supervised learning is typically applied to solve problems where there is a partially labelled data set, for example where only a subset of the data is labelled. Semi-supervised machine learning makes use of externally provided labels and objective functions as well as any implicit data relationships. When initially configuring a machine learning system, particularly when using a supervised machine learning approach, the machine learning algorithm can be provided with some training data or a set of training examples, in which each example is typically a pair of an input signal/vector and a desired output value, label (or classification) or signal. The machine learning algorithm analyses the training data and produces a generalised function that can be used with unseen data sets to produce desired output values or signals for the unseen input vectors/signals. The user needs to decide what type of data is to be used as the training data, and to prepare a representative real-world set of data. The user must however take care to ensure that the training data contains enough information to accurately predict desired output values without providing too many features, which can result in too many dimensions being considered by the machine learning process during training and could also mean that the machine learning process does not converge to good solutions for all or specific examples. The user must also determine the desired structure of the learned or generalised function, for example whether to use support vector machines or decision trees.

Developing a machine learning system typically consists of two stages: (1) training and (2) production. During the training the parameters of the machine learning model are iteratively changed to optimise a particular learning objective, known as the objective function or the loss. Once the model is trained, it can be used in production, where the model takes in an input and produces an output using the trained parameters. During training stage of neural networks, verified inputs are provided, and hence it is possible to compare the neural network's calculated output to then the correct the network is need be. An error term or loss function for each node in neural network can be established, and the weights adjusted, so that future outputs are closer to an expected result. Machine learning may be performed through the use of one or more of: a non-linear hierarchical algorithm; neural network; convolutional neural network; recurrent neural network; long short-term memory network; multi-dimensional convolutional network; a memory network; fully convolutional network or a gated recurrent network allows a flexible approach when generating the predicted block of visual data. The use of an algorithm with a memory unit such as a long short-term memory network (LSTM), a memory network or a gated recurrent network can keep the state of the predicted blocks from motion compensation processes performed on the same original input frame. The use of these networks can improve computational efficiency and also improve temporal consistency in the motion compensation process across a number of frames, as the algorithm maintains some sort of state or memory of the changes in motion. This can additionally result in a reduction of error rates.

Any system features as described herein may also be provided as method features, and vice versa. As used herein, means plus function features may be expressed alternatively in terms of their corresponding structure.

Any feature in one aspect may be applied to other aspects, in any appropriate combination. In particular, method aspects may be applied to system aspects, and vice versa. Furthermore, any, some and/or all features in one aspect can be applied to any, some and/or all features in any other aspect, in any appropriate combination.

It should also be appreciated that particular combinations of the various features described and defined in any aspects of the invention can be implemented and/or supplied and/or used independently.

What is claimed is:

1. A computer-implemented method of prompting a user based on a determined mental state of a user, the method comprising the steps of:
receiving user biometric data;
determining at least one mental state of the user based on the received user biometric data; and
on determining the at least one mental state of the user is a predetermined mental state, outputting one or more dynamic prompts for display to the user based on the at least one mental state of the user.

2. The method of claim 1, wherein the step of receiving user biometric data comprises receiving user biometric data from any or any combination of: a wearable device of the user; images of the user; user speech data; or user text data.

3. The method of claim 1, further comprising the step of receiving user response data entered by the user in response to the one or more dynamic prompts.

4. The method of claim 1, wherein the at least one mental state of the user comprises any one or more of: at least one emotional state; a range of emotional state; a range of emotional states; a range of mental state; a range of mental states; a probability score, optionally of a given mental state and/or emotional state; a confidence value, optionally of a given mental state and/or emotional state; one or more confidence values, optionally of a given mental state and/or emotional state; a probability distribution of confidence values, optionally of a given mental state and/or emotional state; one or more clinical scores of emotional state; one or more clinical scores of mental state; one or more clinical scores of mental illness; a PHQ-9 score; a GAD-7 score; one or more cognitive markers of depression and/or anxiety; any other clinical measure(s) of mental illness.

5. (canceled)

6. (canceled)

7. The method of claim 1, wherein the at least one mental state of the user is determined using a computer based model:
optionally the computer based model comprising one or more machine learning algorithms, wherein the one or more machine learning algorithms process any or any combination of: user biometric data; supplementary data; continuous data; speech; and/or text.

8. (canceled)

9. (canceled)

10. (canceled)

11. The method of claim 1, wherein the user biometric data comprises supplementary and/or continuous data, and optionally, wherein the supplementary and/or continuous data is received in substantially real-time.

12. The method of claim 11, wherein the supplementary data is received from a secondary user device and wherein the supplementary data comprises any one or more of: mobile data; geo location data;
mobile usage data; typing speed; er-accelerometer data and/or wherein the continuous data comprises any one or more of: heart data; peripheral skin temperature data; Galvanic Skin Response (GSR) data; location data.

13. (canceled)

14. The method of any claim 1, wherein the user biometric data further comprises any one or more of: sleep data; activity data;
historical emotional states; historic mental states.

15. The method of claim 1, wherein the one or more dynamic prompts comprise any or any combination of: mood based prompts; time based prompts; location based prompts; people based prompts;
response triggering prompts, optionally wherein the response triggering prompts comprise requesting the user to provide user response data.

16. The method of claim 1, wherein the at least one mental state of the user is substantially discrete:
optionally wherein the at least one mental state of the user comprises any one or more of: happy; sad; pleasure; fear; anger; hostility; calmness; excitement; and/or any other psychological and/or emotional and/or mental state relevant to the mental health of the user.

17. (canceled)

18. (canceled)

19. (canceled)

20. (canceled)

21. (canceled)

22. The method of claim 1, wherein the step of determining at least one mental state of the user based on the received user biometric data further comprises determining at least one associated confidence value, and optionally wherein the step of outputting one or more dynamic prompts for display to the user based on the at least one mental state of the user comprises outputting one or more dynamic prompts for display to the user based on the at least one mental state of the user and one of the at least one associated confidence values.

23. (canceled)

24. The method of claim 1, further comprising using one or more probabilistic models, and optionally wherein any of the steps of:
(a) determining at least one mental state of the user based on the received user biometric data; and/or (b) on determining the at least one mental state of the user is a predetermined mental state, outputting one or more dynamic prompts for display to the user based on the at least one mental state of the user;
comprise using one or more probabilistic models.

25. (canceled)

26. The method of claim 12, wherein the one or more probabilistic models comprise any or any combination of: a Bayesian deep neural network incorporating Monte Carlo dropout or variational Bayes methods to approximate a probability distribution over one or more outputs; a hidden Markov model; a Gaussian process; a naïve Bayes classifier; a probabilistic graphical model; a linear discriminant analysis model; a latent variable model; a Gaussian mixture model; a factor analysis model; an independent component analysis model; and/or any other probabilistic machine learning method/technique that generates a probability distribution over its output.

27. The method of any of claim 11, wherein the step of outputting one or more dynamic prompts for display to the user based on the at least one mental state of the user is dependent on a combination of the at least one mental state of the user and the at least one associated confidence values of the at least one mental state, and optionally the at least one associated confidence values of the at least one mental states exceeds one or more predetermined thresholds.

28. (canceled)

29. The method of claim 3, wherein the user response data comprises any or any combination of: information for use in a clinical setting; clinically meaningful data from the user, optionally comprising any or any combination of associated thoughts, feelings and/or behaviours; data gathered at clinically salient moments; data gathered within a predetermined time of the detected at least one mental states; associated relevant data, optionally comprising one or more associated confidence values and optionally wherein the associated relevant data is used to assign a weighting and/or importance to the user response data.

30. (canceled)

31. The method of claim 15, further comprising a step of performing statistical analysis on the relationships between the user response data and the determined at least one mental states, optionally outputting one or more measures of the relationships between the user response data and the determined at least one mental states.

32. The method of claim 15, wherein outputting one or more dynamic prompts for display to the user comprises outputting one or more dynamic prompts supplying content to the user determined to be relevant to the determined at least one mental state of the user; and wherein the user response data is collected following the content being supplied to the user.

33. A method for prompting a user based on a determined at least one mental state of the user, wherein the at least one mental state of the user is determined using user biometric data, the method comprising the steps of:
receiving one or more dynamic prompts from a server system, wherein the one or more dynamic prompts is based on at least one predetermined mental state of the user;
notifying a user with the one or more dynamic prompts.

34. The method of claim 33, further comprising one or more user interfaces, wherein the one or more user interfaces displays one or more of: the at least one pre-determined mental state of the user; at least one pre-determined emotional state of the user; user biometric data comprising at least heart data; the one or more user inputs; the one or more dynamic prompts; the mental state of the user; and/or the emotional state of the user.

35. The method of claim 33, further comprising a step of determining one or more recommendations based on the determined mental state of the user or further comprising the step of training a computer-based model for determining the mental state of the user based on any one or more of: the user biometric data; the one or more user inputs; the mental state of the user; the emotional state of the user; the at least one pre-determined mental state of the user; and/or the at least one pre-determined emotional state of the user.
